(19) United States
(12) Patent Application Publication
(10) Pub. No.: US 2018/0161002 A1
(43) Pub. Date: Jun. 14, 2018

(54) ULTRASOUND DELIVERY FOR DIAGNOSIS AND/OR THERAPY

(71) Applicant: Medtronic, Inc., Minneapolis, MN (US)

(72) Inventors: Jamu K. Alford, Simi Valley, CA (US); Erik R. Scott, Maple Grove, MN (US); John R. LaLonde, Lake Elmo, MN (US); Yohan Kim, Fridley, MN (US); Jason E. Agran, Gainesville, FL (US)

(73) Assignee: Medtronic, Inc., Minneapolis, MN (US)

(21) Appl. No.: 15/580,940

(22) PCT Filed: Apr. 28, 2016
(86) PCT No.: PCT/US2016/029867

§ 371 Date: Dec. 8, 2017

Related U.S. Application Data
(60) Provisional application No. 62/191,135, filed on Jul. 10, 2015.

(51) Int. Cl. A61B 8/00; A61B 5/01; A61B 8/08; A61N 7/00

(52) U.S. Cl. CPC A61B 8/4236; A61B 5/015; A61N 7/00; A61B 8/48; A61B 8/5223; A61B 8/085

(57) ABSTRACT

In some examples, a system includes one or more ultrasound transducers, one or more temperature sensors, a user interface, and one or more processors. The one or more processors are configured to control the one or more ultrasound transducers to deliver ultrasound to a target point of tissue of a patient to heat the target point of tissue, control the one or more temperature sensors to sense a temperature of other tissue of the patient proximate to the target point of tissue a plurality of times over a period of time after the target point of tissue has been heated, and present, via the user interface, information indicating flow of heat from the target point of tissue to the other tissue over the period of time based on the sensed temperatures to facilitate characterization of at least one of anatomy or function of the tissue.

This application claims the benefit of priority from U.S. Provisional Application Ser. No. 62/191,135, filed Jul. 10, 2015, the entire contents of which are incorporated herein by reference.

### TECHNICAL FIELD

This disclosure relates to delivery of ultrasound for diagnosis and/or therapy.

### BACKGROUND

Delivery of ultrasound involves delivering sound waves with frequencies higher than the upper audible limit of human hearing. Delivery of ultrasound is performed for diagnostic imaging, e.g., to visualize internal body structures such a tendons, muscles, joints, vessels, and internal organs. Ultrasound images are made by delivering ultrasound, e.g., pulses of ultrasound energy, into tissue using one or more ultrasound transducers. The sound echoes, or reflects, off the tissue, with different tissues having different characteristics reflecting the sound differently. The reflected sound is sensed by one or more ultrasound transducers.

Ultrasound has also been delivered to patients for therapeutic purposes. For example, ultrasound has been delivered to promote healing and/or blood flow. As another example, ultrasound has been delivered to modify or destroy problematic tissue, such as tumors. In both cases, the therapeutic effect of ultrasound may be due to heating and/or cavitation of the tissue.

Delivery of ultrasound for medical purposes often involves a relatively-large, cart-based piece of equipment that includes, for example, circuitry for generating and sensing ultrasound signals, processing circuitry, a user interface, and an internal power source and/or the ability to be plugged to AC mains power. A probe that includes the one or more ultrasound transducers may be connected to ultrasound device by a cable.

### SUMMARY

This disclosure is related to devices, systems, and techniques for delivery of ultrasound for diagnosis and/or therapy. In some examples, the disclosure describes techniques for characterizing anatomy and/or function of tissue by delivery of ultrasound to heat tissue and sensing the flow of heat in the tissue after the delivery of ultrasound. The anisotropic heat flow over time may indicate structural and functional characteristics of the tissue. In some examples, a flexible device, e.g., a patch, capable of being attached to a patient for delivery of ultrasound to tissue of the patient includes a plurality of ultrasound transducers, at least one power source, and signal generation and processing circuitry.

In one example, this disclosure is directed to a system comprising one or more ultrasound transducers, one or more temperature sensors, a user interface, and one or more processors. The one or more processors are configured to control the one or more ultrasound transducers to deliver ultrasound to a target point of tissue of a patient to heat the target point of tissue, control the one or more temperature sensors to sense a temperature of other tissue of the patient proximate to the target point of tissue a plurality of times over a period of time after the target point of tissue has been heated, and present, via the user interface, information indicating flow of heat from the target point of tissue to the other tissue over the period of time based on the sensed temperatures to facilitate characterization of at least one of anatomy or function of the tissue.

In another example, this disclosure is directed to a method for facilitating characterization of at least one of anatomy or function of tissue of a patient. The method comprises delivering, using one or more ultrasound transducers, ultrasound to a target point of the tissue to heat the target point of the tissue, sensing, using one or more temperature sensors, a temperature of other tissue proximate to the target point of the tissue a plurality of times over a period of time after the target point of the tissue has been heated, and presenting, via the user interface, information indicating flow of heat from the target point of the tissue to the other tissue over the period of time based on the sensed temperatures to facilitate the characterization of at least one of anatomy or function of the tissue.

In another example, this disclosure is directed to a system comprising means for performing any of the methods described in this disclosure.

In another example, this disclosure is directed to a computer-readable storage medium comprising instructions that, when executed by one or more processors, cause the one or more processors to perform any of the methods described in this disclosure.

In another example, this disclosure is directed to a device configured to deliver ultrasound to tissue of a patient, the device comprising a flexible interconnect element, a plurality of ultrasound transducers distributed on and connected to the flexible interconnect element, one or more power sources connected to the flexible interconnect element, and signal generation circuitry powered by the one or more power sources and connected to the flexible interconnect element. The device further comprises one or more processors powered by the one or more power sources and connected to the flexible interconnect element, wherein the one or more processors are configured to control the signal generation circuitry apply at least one signal to a selected one or more of the plurality of ultrasound transducers and thereby control the one or more ultrasound transducers to deliver ultrasound to the tissue of the patient. The device further comprises an attachment element configured to attach the device to the patient, wherein attachment element is connected to at least one of the flexible interconnect element, the plurality of ultrasound transducers, the one or more power sources, the signal generation circuitry, or the one or more processors.

The details of one or more examples of this disclosure may be set forth in the accompanying drawings and the description below. Other features, objects, and advantages of this disclosure may be apparent from the description and drawings, and from the claims.

### BRIEF DESCRIPTION OF DRAWINGS

FIG. 1 is a conceptual diagram illustrating an example system for delivering ultrasound to a patient.

FIGS. 2A and 2B are top-view and side-view diagrams, respectively, illustrating an example wearable ultrasound device.

FIG. 3 is a top-view diagram illustrating another example wearable ultrasound device.

FIG. 4 is a top-view diagram illustrating another example wearable ultrasound device that includes a plurality of temperature sensors.

FIG. 5 is a functional block diagram illustrating an example configuration of a wearable ultrasound device.

FIG. 6 is a functional block diagram illustrating an example configuration of an interface device configured to communicate with a wearable ultrasound device.

FIG. 7 is a conceptual diagram illustrating an ultrasound actuator in conjunction with patient tissue according to an example technique for structural and/or functional characterization of the tissue based on heat flow.

FIG. 8 is a conceptual diagram illustrating delivery of ultrasound to a target point of the tissue according to the example technique for structural and/or functional characterization of the tissue based on heat flow.

FIG. 9 is a conceptual diagram illustrating flow of heat from the target point to other tissue according to the example technique for structural and/or functional characterization of the tissue based on heat flow.

FIG. 10 is a conceptual diagram illustrating an example anatomical and/or functional map of the tissue determined according to the example technique for structural and/or functional characterization of the tissue based on heat flow.

FIG. 11 is a flow diagram illustrating an example method for delivering ultrasound to tissue for structural and/or functional characterization of the tissue based on heat flow.

### DETAILED DESCRIPTION

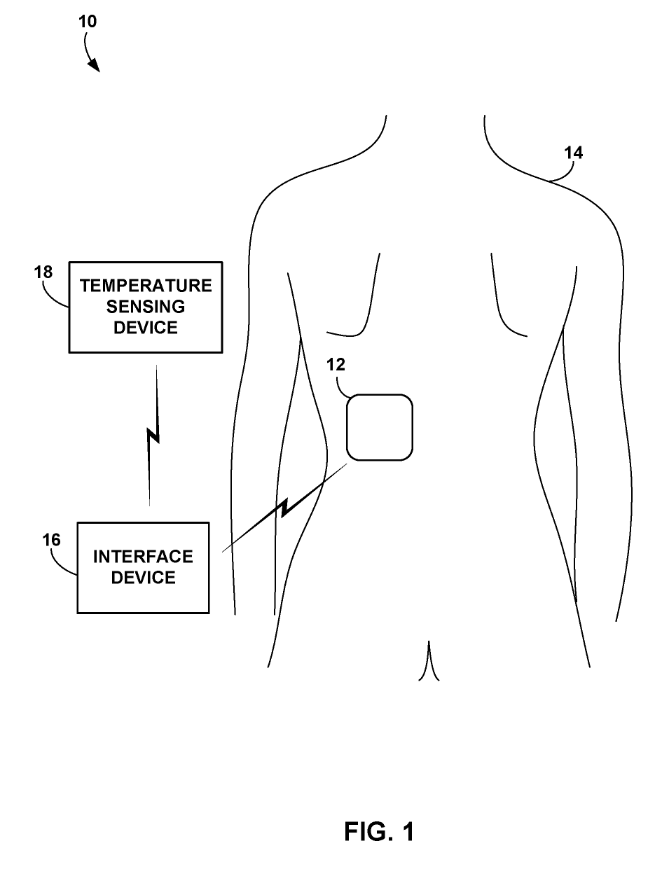

FIG. 1 is a conceptual diagram illustrating an example system 10 for delivering ultrasound to a patient 14. As illustrated in FIG. 1, system 10 includes a wearable ultrasound device 12 attached to patient 14. As will be described in greater detail below, wearable ultrasound device 12 may include a plurality of ultrasound transducers, signal generation circuitry configured to drive the plurality of ultrasound transducers, one or more power sources configured to power the signal generation circuitry, and one or more processors configured to control the signal generation circuitry.

In some examples, the components of wearable ultrasound device 12 may be configured, e.g., constructed and arranged, such that wearable ultrasound device 12 is flexible. In some examples, wearable ultrasound device 12 is flexible such that it conforms to a surface of patient 14 on which the wearable ultrasound device is attached. Wearable ultrasound device 12 may be used, and attached to patient 14, for time periods as brief as a few minutes to as long as several months. The flexibility of wearable ultrasound device 12 may increase the comfort of patient 14.

System 10 may be used for diagnostic and/or therapeutic applications, and may include an attachment element configured to maintain the position of the ultrasound transducers of wearable ultrasound device 12 relative to a treatment or diagnostic area of patient 14. In some examples, wearable ultrasound device 12 may include an adhesive layer as an attachment element for attaching the device to patient 14. In addition to, or instead of the adhesive layer, in some examples, an attachment element may comprise a strap or garment.

Wearable ultrasound device 12 may deliver ultrasound to patient 14 for diagnostic imaging. In some examples, wearable ultrasound device 12 may deliver ultrasound to patient 14 for therapeutic purposes, such as tissue modification, e.g., wound healing or therapeutic tissue destruction, or neuromodulation. In some examples, while delivering ultrasound for a therapeutic purpose, ultrasound device 12 may also image tissue of patient 14, e.g., for visualization of a target region, monitoring temperature and/or cavitation to evaluate therapy effectiveness and patient safety, or beam aberration correction. Ultrasound device 12 may image during delivery of ultrasound based on reflection of the therapeutic ultrasound by tissue of patient 14, or by interleaving delivery of therapeutic ultrasound with imaging ultrasound. The location of wearable ultrasound device 12 on patient 14 illustrated in FIG. 1 is merely one example, and wearable ultrasound device 12 may be attached anywhere on patient 14 to facilitate a particular diagnostic or therapeutic function.

In some examples, as will be described in greater detail below, wearable ultrasound device 12 may deliver ultrasound to tissue of patient 14 to heat a target point of the tissue. The flow or propagation of heat, e.g., the rate of heat flow, from the target point to other tissue of patient 14 proximate to the target point may facilitate characterization of at least one of the anatomy or function of the tissue. Such characterization may allow identification of structures, such as tumors or lesions, and may guide treatment of such structures.

Thermal propagation through patient 12 may be affected by multiple factors, including active mechanisms, such as adaptive blood flow, and passive mechanisms, such as tissue conduction and convection. In general, heat will flow at different rates through tissues having different characteristics, such as different density or vascularization. Heat may flow at a different rate through a blood vessel, tumor, lesion, organ, or lymph node or duct, than through tissues surrounding such structures. Heat may also flow at a different rate through damaged or diseased tissue than tissue that is not damaged or diseased. The heat flow, e.g., the anisotropic nature of the heat flow, from the target point to the proximate tissue may reveal the structure and/or function of the tissue. Accordingly, the heat flow may facilitate diagnosis of numerous conditions, such as diabetes or tumors.

In order to determine the heat flow from the target point to the proximate tissue, system 10 may sense temperatures of the tissue proximate the target point plurality of times over a period of time that begins after the target point has been heated. Reflection of delivered ultrasound by a particular tissue varies based on the temperature of the tissue and, consequently, the temperature of tissue can be sensed via ultrasound imaging of the tissue. In some examples, the ultrasound transducers of wearable ultrasound device 12 sense the temperature of the proximate tissue. In some examples, system 10 includes another ultrasound device to sense the temperature of the surrounding tissue based on ultrasound imaging of the proximate tissue, or another ultrasound device heats the target point and wearable ultrasound device 12 senses the resulting temperature of proximate tissue. Using ultrasound to sense the temperature of the proximate tissue may facilitate sensing temperature of tissue below the outer surface of patient 14, e.g., a three-dimensional volume of tissue surrounding the target point.

In some examples, wearable ultrasound device 12, or another device of system 10, includes temperatures sensors of any type capable of sensing temperature of tissue. For example, wearable ultrasound device 12 may include one or more temperature sensors, such as thermistors or thermocouples, to sense the temperature of tissue proximate to the target point, e.g., at the skin surface of patient 14. As another example, system 10 may include a temperature sensing device 18 that is separate from wearable ultrasound device 12, and includes one or more temperature sensors configured to sense the temperature of tissue proximate to the target point. In some examples, temperature sensing device 18 may include one or more thermal imaging devices, such as infrared cameras or thermometers, to sense the temperature of tissue proximate to the target point.

As illustrated in FIG. 1, system 10 also includes an interface device 16, which may be a computing device having a user interface, e.g., a personal computer, workstation, tablet computing device, or cellular telephone. Interface device 16 is configured to communicate, e.g., via a wired or wireless connection, with wearable ultrasound device 12. Interface device 16 may also be configured to communicate, e.g., via a wired or wireless connection, with temperature sensing device 18. Interface device 16 may control, e.g., program, wearable ultrasound device 12 and temperature sensing device 18. Interface device 16 may also receive sensed information from wearable ultrasound device 12 and temperature sensing device 18, such as sensed temperatures and/or ultrasound images. Although not illustrated in FIG. 1, system 10 may include one or more other remote computing devices connected to interface device 16 via a network, and the one or more remote computing devices may control and/or receive information from wearable ultrasound device 12 and temperature sensing device 18 via interface device 16. In some examples, interface device 16 and temperature sensing device 18 may be integrated as a single device.

System 10 includes one or more processors, e.g., of wearable ultrasound device 12, interface device 16, temperature sensing device 18, and/or the one or more remote computing devices, that are configured to control wearable ultrasound device 12, interface device 16, temperature sensing device 18, or any other ultrasound device, temperature sensing device, or any other device described herein to provide the functionality described herein. For example, one or more processors of one or more of these devices may be configured to control one or more ultrasound transducers, e.g., of wearable ultrasound device 12, to deliver ultrasound, e.g., for imaging, therapy, or to a target point of tissue of a patient to heat the target point of tissue. As another example, the one or more processors of one or more processors of one or more of these devices may be configured to control one or more temperature sensors, e.g., of wearable ultrasound device 12 or temperature sensing device 18, to sense temperature, e.g., to prevent overheating during therapeutic ultrasound, or to sense a temperature of other tissue of patient proximate 14 to the target point of tissue during a period of time after the target point of tissue has been heated. Based on the sensed temperatures, the one or more processors of one or more of these devices may present to a user, e.g., via the user interface of interface device 16 or a remote computing device, information indicating flow of heat from the target point of tissue to the other tissue over the period of time to facilitate characterization of at least one of anatomy or function of the tissue by the user, as will be described in greater detail below.

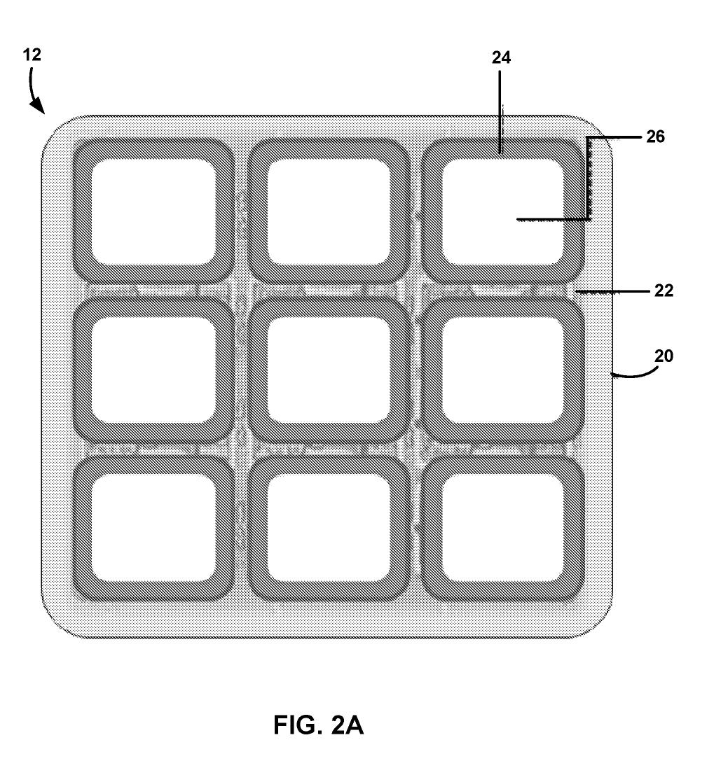

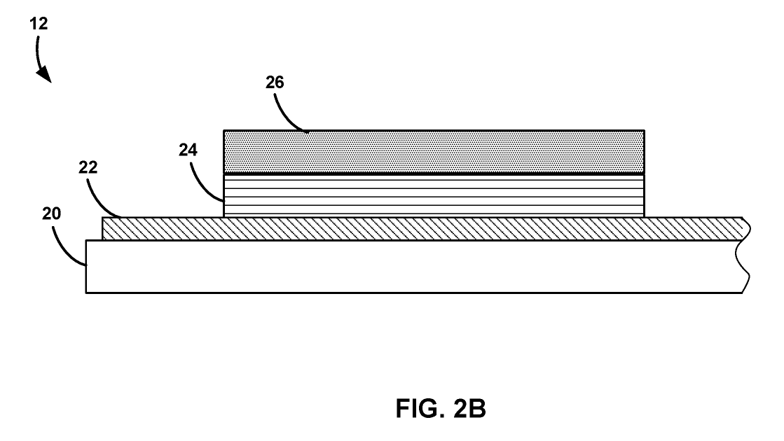

FIGS. 2A and 2B are top-view and side-view diagrams, respectively, illustrating one example configuration of wearable ultrasound device 12. In the example of FIGS. 2A and 2B, wearable ultrasound device 12 includes an adhesive layer 20, a flexible interconnect element 22, a plurality of ultrasound transducers 24 connected to flexible interconnect element 22, and a plurality of power sources 26, e.g., batteries, connected to flexible interconnect element 22. FIG. 2B illustrates one ultrasound transducer 24 and one battery 26. Although not illustrated in FIGS. 2A and 2B, wearable ultrasound device 12 may also include signal generation circuitry, one or more processors, sensing circuitry, and communication circuitry, e.g., configured to communicate with interface device 16 (FIG. 1), connected to flexible interconnect element 22.

The components of wearable ultrasound device 12 may be configured, e.g., constructed and arranged, such that wearable ultrasound device 12 is flexible. For example, flexible interconnect element 22 may comprise a flexible circuit, e.g., a flex circuit that electrically connects two or more of the components of wearable ultrasound device 12. Flexible interconnect element 22 and adhesive layer 20 may comprise mechanically compliant materials. Additionally, ultrasound transducers 24 and power sources 26 may be discrete and distributed across wearable ultrasound device 12, e.g., in a two-dimensional array as illustrated in FIG. 2A, which may facilitate flexibility of the wearable ultrasound device. In some examples, signal generation circuitry that drives ultrasound transducers 24 may include flexible driving electronics.

Having a plurality of power sources 26 may also increase the onboard power capacity of wearable ultrasound device 12. In some examples, power sources 26 comprise rechargeable batteries. In such examples, wearable ultrasound device 12 may include a recharge interface, such as a coil for inductive recharging or connector, e.g., universal serial bus (USB), mini-USB, or micro-USB, for wired recharging of power sources 26. In some examples, interface device 16 (FIG. 1) or another device charges power sources 26 of wearable ultrasound device 12,

In some examples, as illustrated by FIG. 2A, each of power sources 26 is associated with a respective one of ultrasound transducers 24. In some examples, each of power sources 26 is attached to the respective ultrasound transducer 24. In such examples, power sources 26 may be configured as a backing material for transducers 24, to tune a frequency of the respective ultrasound transducer. Some ultrasound systems may include a backing material behind the acoustic material to ‘tune’ the frequency. Using power sources 26 as a backing material may reduce or eliminate the need for a dedicated backing material to tune transducers 24, which may in turn reduce the size, e.g., volume, thickness, or weight of the transducers. Various features of power sources 26, such as thickness and mass, may be chosen to tune the ultrasound output parameters, e.g., frequency. In some examples, flexible interconnect element 22 may also be configured as a backing material for ultrasound transducers 24, in addition to, or instead of, power source 26. In some examples, additional electrical components may be affixed, e.g., directly, to the ultrasound material, e.g., during the manufacturing process, and may act as backing material for transducers 24, alone or in combination with other components of device 12.

The relative vertical arrangement of adhesive layer 20, interconnect layer 22, ultrasound transducers 24, and power sources 26 illustrated in FIG. 2B is merely one example. In other examples, interconnect layer 22 may be at least partially between ultrasound transducers 24 and power sources 26, or power sources 26 may be at least partially between interconnect layer 22 and ultrasound transducers 24. In some examples, discrete components, such as ultrasound transducers 24 and power sources 26, may be located at least partially within, e.g., may be at least partially surrounded by, interconnect layer 22 and/or adhesive layer.

Although nine ultrasound transducers 24 and nine power sources 26 are illustrated in FIG. 2A, in other examples, the numbers of transducers and power sources may be different than illustrated, and the number of transducers 24 may be different than the number of power sources. In some examples, there may be at least three, at least nine, at least thirty-two, or at least sixty-four transducers 24 and/or power sources 26. In some examples, power sources 26 may be horizontally adjacent transducers 24. In some examples, one or more power sources 26 may be located anywhere in interconnect element 22 to power transducers 24 (e.g., the signal generation circuitry that drives the transducers) and other components of wearable ultrasound device 12.

Power sources 26 may be connected in series, parallel or in some series/parallel combination. At least partial series combination may boost voltage of the resulting power source. To improve acoustic coupling and tune the ultrasound, the cavity within the power source case (e.g., a battery case) may substantially free of gas (e.g., free or nearly free), such as by completely filling the space between electrodes with an electrolyte that may be liquid, gel or solid. In some examples, power sources 26 comprise a battery chemistry that does not generate gas during charge/discharge (for example, using a lithium titanate anode) and/or to allow for removal of gas that is usually formed during the initial charge cycle (known in the art as formation) of the cell. The power source encasement may be a metal such as titanium or aluminum or a metal/polymer foil laminate, although other materials can be used in other examples. The performance of power sources 26 as backing material may be configured based on acoustic impedance (density×sound speed), thickness, and attenuation coefficient to reduce reflections.

The piezoelectric material of ultrasound transducers 24 may be, as examples, one or more of lead zirconate titanate (PZT) composite, PZT film, polyvinylidene fluoride (PVDF), which is a plastic with piezoelectric properties, and/or capacitive micromachined ultrasonic transducers (CMUTs). In examples in which power sources 26 and transducers 24 are attached, the ultrasound material may be glued or otherwise bonded to the surface of the power source. In some examples, a metallic housing of a power source 26 may be part of an electrical circuit of wearable ultrasound device 12, e.g., to couple ultrasound material of a transducer 24 to the power source 26, signal generation circuitry for driving the transducer 24, and/or sensing circuitry for processing reflected ultrasound for diagnostic or therapy monitoring purposes.

Adhesive layer 20 attaches wearable ultrasound device 12 to patient 14 (FIG. 1). In some examples, adhesive layer 20 is also configured to provide an acoustic interface between transducers 24 and tissue of patient 14 for ultrasound. In such examples, the adhesive of adhesive layer 20 may be between, e.g., substantially completely fill the space between, each of transducers 24 and a surface of patient 14.

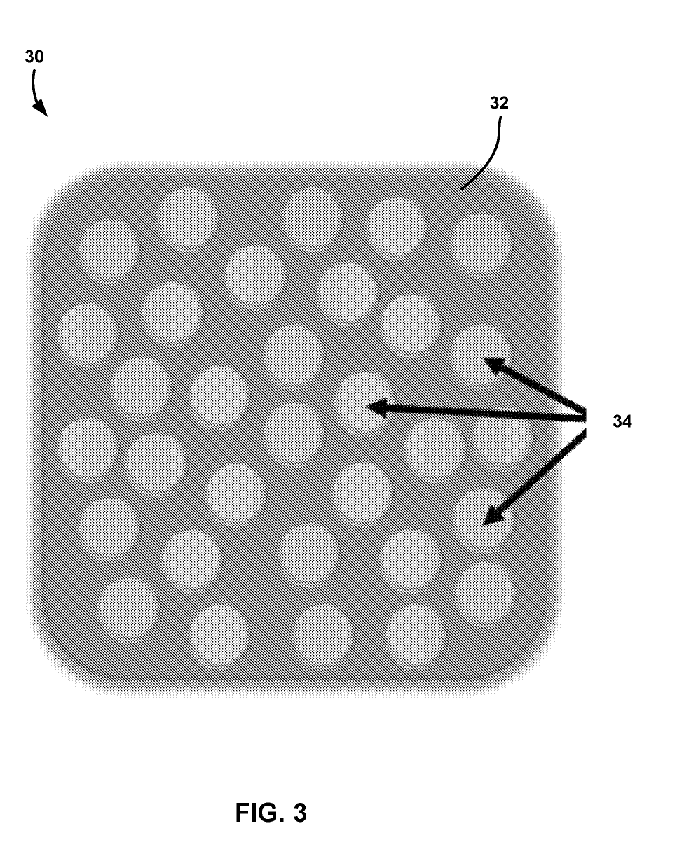

FIG. 3 is a top-view diagram illustrating another example wearable ultrasound device 30. Like wearable ultrasound device 12 of FIGS. 1-2B, wearable ultrasound device 30 includes an interconnect element 32, and a plurality of ultrasound transducers 34 connected to the interconnect element. Although not illustrated in FIG. 3, wearable ultrasound device 30 may also include an adhesive layer, one or more power sources, and other electrical components described above with respect to wearable ultrasound device 12 of FIGS. 1-2B.

Like wearable ultrasound device 12 of FIGS. 1-2B, ultrasound transducers 34 of wearable ultrasound device 30 are distributed on interconnect element 32 in a two-dimensional array. However, unlike ultrasound transducers 24 in FIG. 2A, ultrasound transducers 34 are not necessarily arranged in rows and columns. Ultrasound transducers 34 may be arranged in any suitable way on wearable ultrasound device 30. Ultrasound transducers 34 may be arranged on wearable ultrasound device 30 in a way that, for example, improves ultrasound delivery and/or sensing, e.g., for a particular diagnostic or therapeutic purpose, reduces size and/or increases flexibility of ultrasound device 30, or improves power consumption or heat dissipation by ultrasound device 30.

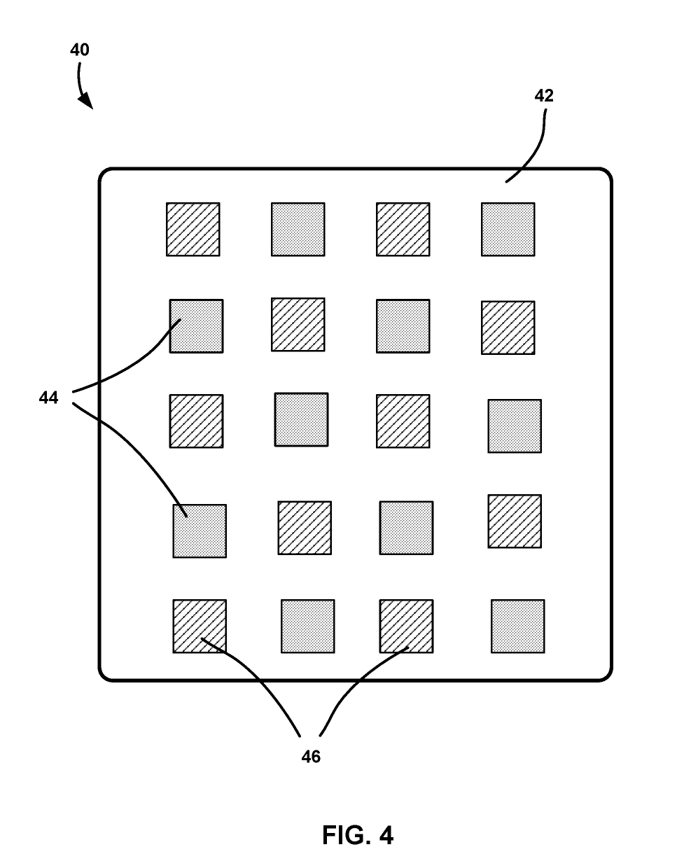

FIG. 4 is a top-view diagram illustrating another example wearable ultrasound device 40. Like wearable ultrasound device 12 of FIGS. 1-2B, wearable ultrasound device 40 includes an interconnect element 42, and a plurality of ultrasound transducers 44 connected to the interconnect element. As illustrated by FIG. 4, wearable ultrasound device 40 additionally includes includes a plurality of temperature sensors 46 connected to interconnect element 42.

Temperatures sensors 46 may be configured to measure temperature at the surface of tissue beneath wearable ultrasound device 40. In some examples, temperature sensors 46 may be dispersed at different locations across wearable ultrasound device 40 to detect temperature at a variety of tissue locations. In the example of FIG. 4, temperature sensors 46 are interspersed among ultrasound transducers 40, but may in other examples be segregated from ultrasound transducers. Temperature sensors 46, and any temperatures sensors described herein, may comprise any circuitry capable of transducing temperature to an electrical signal, such as thermistors, thermocouples, resistance temperature detectors (RTDs), or infrared sensors.

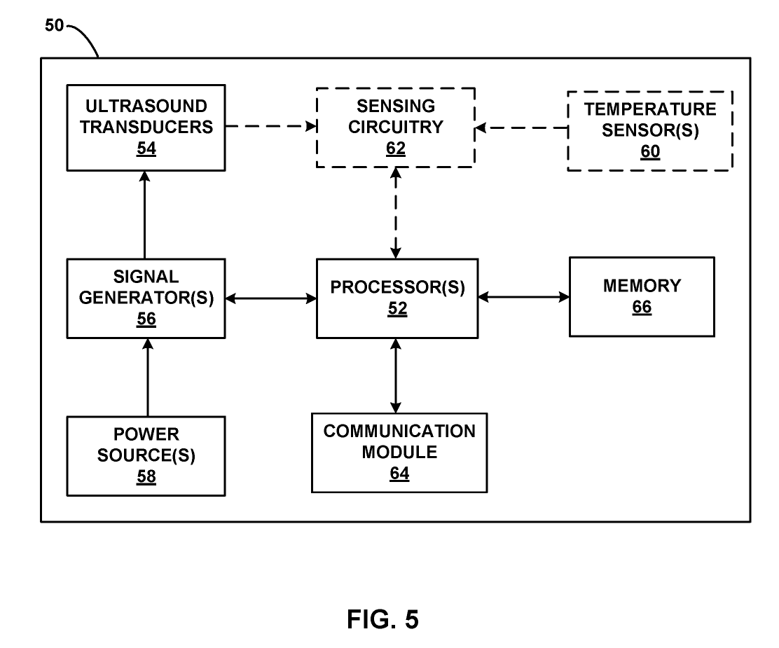

FIG. 5 is a functional block diagram illustrating an example configuration of a wearable ultrasound device 50, which may correspond to any of wearable ultrasound devices 12 (FIGS. 1-2B), 30 (FIG. 3), and 40 (FIG. 4). As illustrated in FIG. 5, ultrasound device 50 includes one or more processors 52, a plurality of ultrasound transducers 54, one or more signal generators 56 for driving the ultrasound transducers 54 to deliver ultrasound, and one or more power sources 58 that provide power to the one or more signal generators 56 for driving transducers 54, as well as providing power to other components ultrasound device 50. Ultrasound transducers 54 and power sources 58 may correspond to any ultrasound transducers, e.g., 24 (FIGS. 2A and 2B), 34 (FIG. 3), or 44 (FIG. 4), and power sources, e.g., power sources 26 (FIGS. 2A and 2B), respectively, described herein.

As illustrated in FIG. 5, ultrasound device 50 may also include a communication module 64 and memory 66. Memory 66, as well as other memories described herein, may include any volatile or non-volatile media, such as a random access memory (RAM), read only memory (ROM), non-volatile RAM (NVRAM), electrically erasable programmable ROM (EEPROM), flash memory, and the like. Memory 66 may store computer-readable instructions that, when executed by processor(s) 52, cause ultrasound device 50 to perform various functions described herein. Processor(s) 52 may comprise any combination of one or more processors including one or more microprocessors, digital signal processors (DSPs), application specific integrated circuits (ASICs), field programmable gate arrays (FPGAs), or other equivalent integrated or discrete logic circuitry. Accordingly, processor(s) 52 may include any suitable structure, whether in hardware, software, firmware, or any combination thereof, to perform the functions ascribed herein to processors (52) and ultrasound device 50.

Processor(s) 52 are configured to control ultrasound transducers 54 to deliver ultrasound, e.g., for a therapeutic or diagnostic purpose. More particularly, processor(s) 52 control signal generator(s) 56 to generate a signal based on power from power source(s) 58 that drives the ultrasound transducers to deliver ultrasound. Signal generator(s) 56 may include one or more oscillators configured to generate signals of a desired frequency for the ultrasound, amplification or other circuitry to control the amplitude of the driving signals, as well as switching circuitry to selectively direct the signal to one or more of transducers 54 and/or selectively control the on/off state of individual ones or groups of transducers 54. Some or all of the signal generation circuitry may be respectively associated with certain ones or groups of transducers 54, or shared by all or a subset of transducers 54. Processor(s) 52 may control ultrasound transducers 54 to deliver ultrasound to a particular depth, region, or point of tissue, with a particular amplitude, by selecting which of transducers 54 is on or driven, and controlling one or more of the amplitude or phase of the driving signal provided to the driven transducers 54 by signal generator(s) 56. Different active transducers 54 may be driven with different signals, e.g., different amplitudes and/or phases, to target a desired, depth, region, or point of tissue.

In examples in which ultrasound device 50 is configured for diagnostic ultrasound, or to sense temperature via diagnostic ultrasound, ultrasound device 50 may include sensing circuitry 62 to selectively, e.g., as controlled by processor(s) 52, receive and condition electrical signals produced ultrasound transducers 54 as a function of reflected ultrasound, for processing by processor(s) 52. Sensing circuitry 62 may include one or more switches to control which one or more of transducers 54 are active to sense reflected ultrasound. In some examples in which ultrasound device 50 is configured to sense temperature, e.g., during delivery of therapeutic ultrasound, or as part of techniques for diagnostic evaluation of tissue based on heat flow over time described in greater detail below, ultrasound device may include one or more temperature sensors 60, which may correspond to any temperature sensors described herein, such as temperatures sensors 46 (FIG. 4). Sensing circuitry 62 may selectively, e.g., as controlled by processor(s) 52, receive and condition electrical signals produced temperature sensor(s) 60 as a function of tissue temperature, for processing by processor(s) 52. Sensing circuitry 62 may include one or more switches to control which one or more of temperature sensor(s) are active to sense temperature.

Power source(s) 58 may deliver operating power to various components of ultrasound device 50. Power source(s) 58 may include a small rechargeable or non-rechargeable batteries and a power generation circuit to produce the operating power. Recharging may be accomplished through proximal inductive interaction between a charging device and an inductive charging coil of ultrasound device 50, or a wired connection between the charging device and ultrasound device 50.

Communication module 64 is configured to support wired or wireless communication between ultrasound device 50 and one or more other devices, such as interface device 16. A user may control the delivery of ultrasound by ultrasound device 50, as well as the collection of diagnostic ultrasound and/or temperature sensing by ultrasound device 50, via communication with processor(s) 52 through communication module 64. In some examples, programs that control the delivery of ultrasound, collection of diagnostic ultrasound, and/or temperature sensing may be stored in memory 66, and executed by processor(s) 52. A user may generate or update such programs, using interface device 16, through communication with ultrasound device 50 via communication module 64. Interface device 16, or another device, may also receive diagnostic ultrasound images or sensed temperatures collected by processor(s) 52, or any other information generated by processor(s) 52, via communication module 64. Such information may be stored in memory 66.

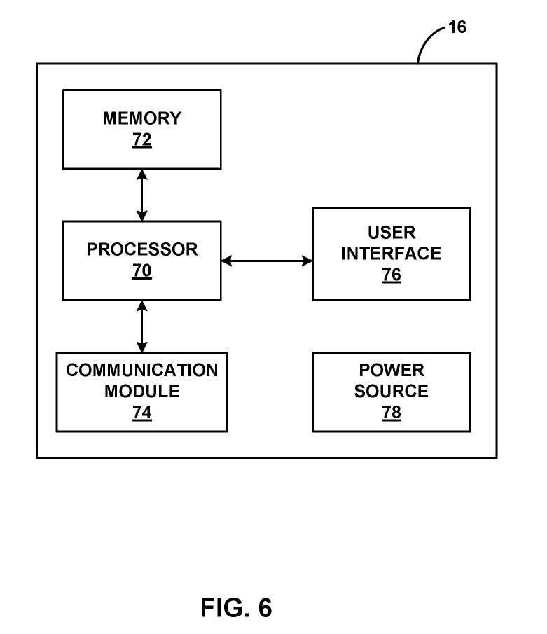

FIG. 6 is a functional block diagram illustrating an example configuration of interface device 16. As illustrated in FIG. 6, interface device 16 includes a processor 70, a memory 72, a communication module 74, a user interface 76, and a power source 78 configured to power the components of interface device 16. Processor 70 controls user interface 76 and communication module 74, and stores and retrieves information and instructions to and from memory 72.

Processor 70 may comprise any combination of one or more processors including one or more microprocessors, DSPs, ASICs, FPGAs, or other equivalent integrated or discrete logic circuitry. Accordingly, processor 70 may include any suitable structure, whether in hardware, software, firmware, or any combination thereof, to perform the functions ascribed herein to processor 70 and interface device 16. Memory 72 may include program instructions that, when executed by processor 70, cause processor 70 and interface device 16 to perform any of the functions ascribed to them herein. Memory 72 may include any volatile or nonvolatile memory, such as RAM, ROM, EEPROM or flash memory.

A user, such as a clinician, other caregiver, or patient 14, may interact with interface device 16 through user interface 76. User interface 76 includes a display, with which processor 70 may present information, such as information relating to heat flow as described in greater detail below, or other information retrieved from ultrasound device 50. In addition, user interface 76 may include an input mechanism to receive input from the user, though which the user may control or program delivery of ultrasound and or sensing of temperature according to any of the techniques described herein. Communication module 74 is configured for wired or wireless communication with the corresponding communication module 64 of ultrasound device 50, to facilitate user control or programming of the ultrasound device, or retrieval of information from the ultrasound device.

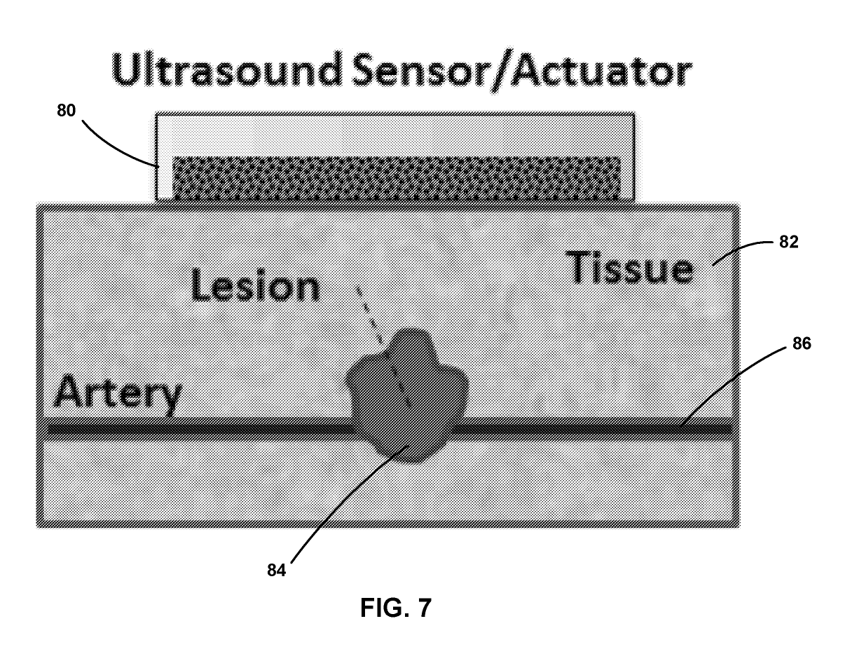

FIG. 7 is a conceptual diagram illustrating an ultrasound actuator and sensing device 80 in conjunction with patient tissue 82 according to an example technique for structural and/or functional characterization of the tissue based on heat flow. Measurement of heat propagation through tissue 82, e.g., the anisotropic rate of heat flow through tissue 82, may facilitate characterization of the anatomy or function of tissue 82.

As illustrated in FIG. 7, tissue may include anatomical structures, such as artery 86, as well as functionally different tissue, such as lesion or tumor 84. Heat may flow differently, e.g., at a different rate, through such anatomically or structurally different tissue. Ultrasound actuator and sensing device 80 may correspond to any of wearable ultrasound devices 12, 30, 40, or 50, or any ultrasound delivery device.

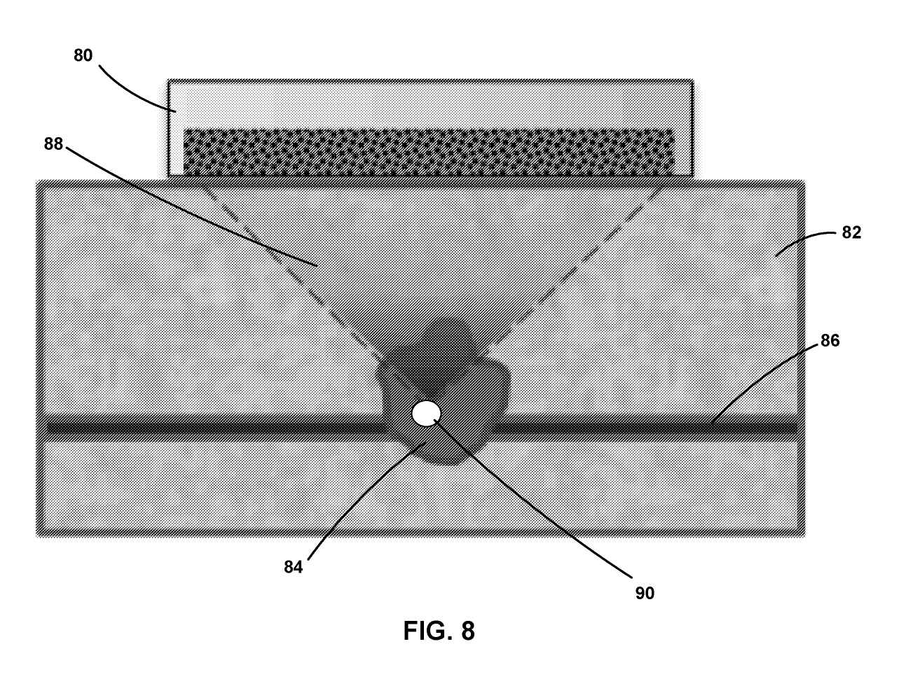

FIG. 8 is a conceptual diagram illustrating delivery of ultrasound to a target point 90 of tissue 82 according to the example technique for structural and/or functional characterization of the tissue based on heat flow. As illustrated in FIG. 8, ultrasound actuator and sensing device 80 may deliver an external, high-intensity focused ultrasound beam 88 to the specific target point 90. One or more processors, e.g., of ultrasound device 80, interface device 16, or any other device described herein, may control the delivery of ultrasound beam 88 focused on point 90 by, for example, controlling one or more of which ultrasound transducers are driven, and respective amplitudes and phases of the signals driving the transducers.

In some examples, ultrasound device 80 delivers ultrasound 88 for a particular length of time, e.g., approximately 1 second to approximately 60 seconds. In some examples, ultrasound device 80 delivers ultrasound 88 until a particular thermal dose, or target temperature of tissue at target point 90, is reached. The target temperature may be, for example, an increase of approximately 0.1 degrees C. to approximately 6 degrees C., such as an increase of approximately 0.5 degrees C. to approximately 6 degrees C. The thermal dose, e.g., temperature at or around target point 90, may be monitored during delivery of ultrasound 88 by thermal ultrasound measurement methods to maintain the temperature below a thermal threshold above which tissue may be adversely impacted, and determine when the target temperature is met. Ultrasound device 80 may monitor the temperature during delivery of ultrasound 88 based on reflected ultrasound, or using one or more temperatures sensors, e.g., temperature sensors 46 or 60 (FIGS. 4 and 5). In other examples, a separate temperature sensing device 18 (FIG. 1) monitors temperature. Once the target tissue has reached the desired temperature, processor(s) 52 control signal generator(s) 56 to halt the sonication, e.g., automatically, or in response input from interface device 16, which may be automatic or manual by a user.

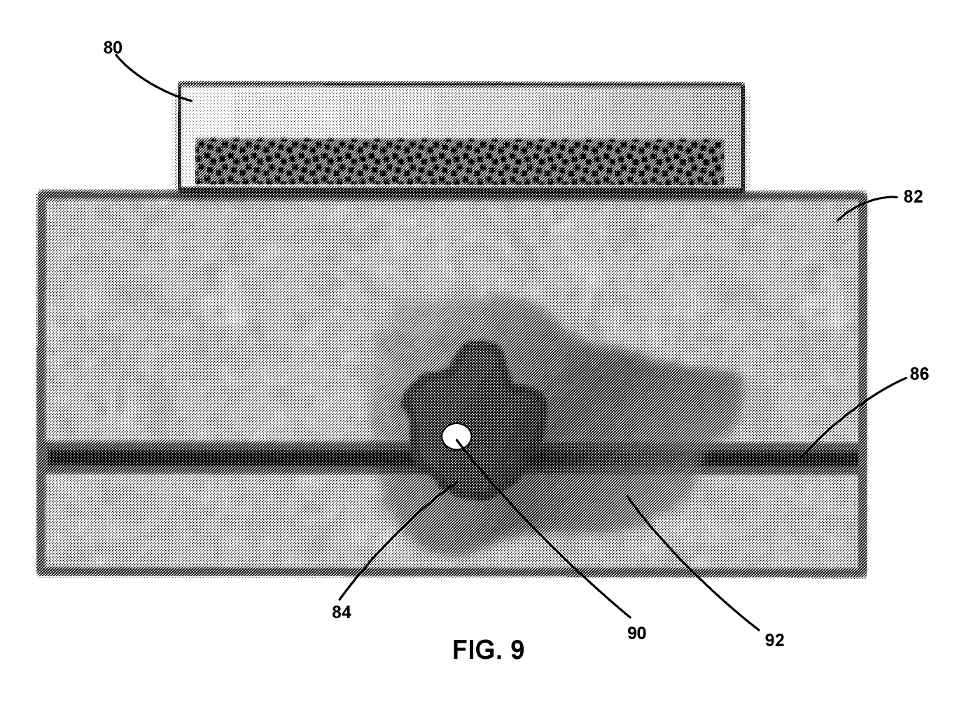

FIG. 9 is a conceptual diagram illustrating flow of heat 92 from target point 90 to other portions of tissue 82 proximate to, e.g., surrounding, target point 90 according to the example technique for structural and/or functional characterization of the tissue based on heat flow. Over a period of several minutes, the heat from the targeted focal zone will spread to the surrounding regions due to thermal transport mechanisms. The ultrasound system (e.g., processor(s) 52, will measure the time course of the temperature flow.

After delivery of heat to target point 90, ultrasound device 80 or temperature sensing device 18 measures temperature a plurality of times over a period of time, e.g., beginning after target point 90 has reached the target temperature. Temperature sensors may be configured, e.g., as an array of ultrasound transducers or other temperature sensors, or otherwise configured, to sense the temperature of a plurality of regions proximate to target point 90. In this manner, the rate of heat flow over time from that point in many or all directions may be measured. In some examples, e.g., when ultrasound transducers are used to measure temperature based on reflected ultrasound, temperatures at respective locations of a three-dimensional volume of tissue proximate the target point may be sensed. In some examples, the temperature measurements over the period of time after heating may be substantially continuous.

Once the heat is substantially fully dissipated, the system including ultrasound device 80 may repeat the measurement using a different target point 90 of tissue 82. By repeating over many target points, it may be possible to develop an anatomical and functional map that could be used to measure tissue health and viability as well as quantify disease state over a three-dimensional volume. In some examples, through a series of measurements, a full three dimensional map is generated, describing the anisotropic heat transfer at all points within the target region. The heat transfer map may provide information about the function and health of the tissue and may provide a novel means to diagnose diseases from diabetes to tumors.

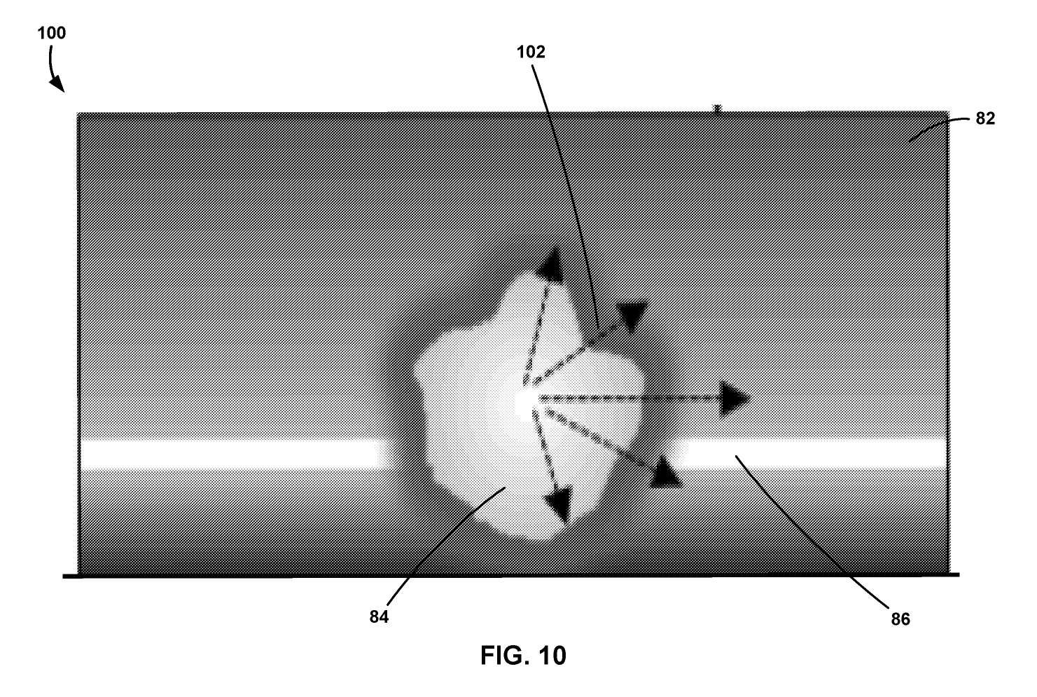

FIG. 10 is a conceptual diagram illustrating an example anatomical and/or functional map 100 of the tissue determined according to the example technique for structural and/or functional characterization of the tissue based on heat flow. Interface device 16 may receive sensed temperature information from ultrasound device 80 and/or temperature sensing device 18 indicating the flow of heat over time from the one or more target points 90. Interface device 16, or another computing device in communication with interface device 16, may generate map 100 based on the received temperature information. As illustrated in FIG. 10, map 100 illustrates tissue 82, including artery 86, and lesion or tumor 84. The lesion or tumor 84 may be revealed by its anisotropic heat flow properties, reflected in the received temperature data. Map 100 may further indicate predominate directions of heat flow, e.g., via arrows 102, which may assist a clinician in characterizing the anatomy or function of tissue 82, such as tumor or lesion 84. In some examples, map 100 includes a plurality of voxels, and indicates thermal diffusion at each of the plurality of voxels. In some examples, interface device 16 or another computing device overlays heat flow data with other anatomical imaging data, such as X-ray, MRI, or CT data, to generate map 100.

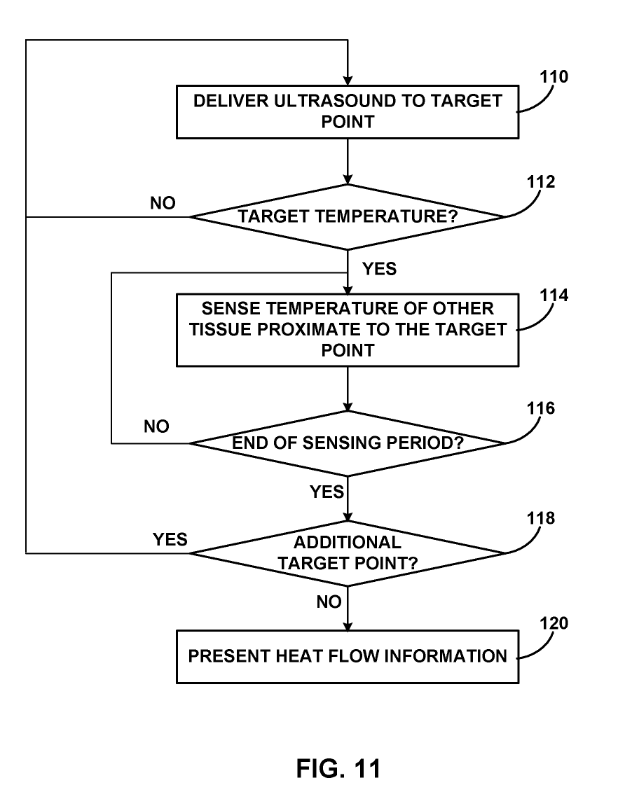

FIG. 11 is a flow diagram illustrating an example method for delivering ultrasound to tissue for structural and/or functional characterization of the tissue based on heat flow. The example method of FIG. 11 may be performed by any system including one or more ultrasound transducers to deliver ultrasound to heat tissue, one or more temperature sensors, and one or more processors to control the delivery of ultrasound and sensing of temperature, and process the sensed temperature to present diagnostic information indicative of the heat flow, e.g., map 100, via a user interface. The example method may be performed by system 10, any system include ultrasound devices 12, 30, 40, 50, or 80. In some examples, the example method of FIG. 11 may be performed by a cart-based ultrasound system configured to sense temperature.

According to the example method of FIG. 11, one or more ultrasound transducers deliver ultrasound to a target point of tissue 90 of a patient (110) until the target point of tissue has reached a target temperature or temperature range (112). When the target point of tissue has reached the target temperature (YES branch of block 112), one or more temperature sensors repeatedly sense temperature of other tissue proximate to the target point (114) until the end of a sensing period (116).

When the sensing period has ended (YES of 116), one or more processors 52 determine whether there are additional target points to test (118). If there are additional target points (YES of 118), the one or more processors control the ultrasound transducers to deliver ultrasound to the next target point until the target temperature is reached, and then control temperature sensors to sense temperature of the other tissue proximate to the next target point for another sensing period (110-116). If there are no additional target points (NO of 118), the system, e.g., interface device 16 or another computing device, may present heat flow information, e.g., map 100, to a user based on the temperatures sensed at tissue proximate to the one or more target points over their respective post-heating sensing periods (120).

The techniques described in this disclosure, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general-purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

A first example includes a method for facilitating characterization of at least one of anatomy or function of tissue of a patient, the method comprising: delivering, using one or more ultrasound transducers, ultrasound to a target point of the tissue to heat the target point of the tissue; sensing, using one or more temperature sensors, a temperature of other tissue proximate to the target point of the tissue a plurality of times over a period of time after the target point of the tissue has been heated; and presenting, via the user interface, information indicating flow of heat from the target point of the tissue to the other tissue over the period of time based on the sensed temperatures to facilitate the characterization of at least one of anatomy or function of the tissue.

A second example includes the method of the first example, wherein the target point of tissue comprises a first target point of tissue, and the method comprises, iteratively, for each of a plurality of target points of tissue including the first target point of tissue: delivering, using the one or more ultrasound transducers, ultrasound to one of the plurality of target points of tissue to heat the target point of tissue; and sensing, using the one or more temperature sensors, a temperature of other tissue of the patient proximate to the target point of tissue a plurality of times over a period of time after the target point of tissue has been heated, and wherein presenting information indicating flow of heat comprises presenting information indicating flow of heat from the plurality of target points of tissue to the other tissue over the periods of time based on the sensed temperatures to facilitate characterization of the at least one of anatomy or function of the tissue.

A third example includes the method of the first example or the second example, wherein delivering ultrasound comprises delivering ultrasound to the target point of tissue until the target point of tissue is heated to a target temperature, and wherein sensing temperature comprises sensing temperature of the other tissue the plurality of times over the period of time after the target point of tissue has been heated to the target temperature.

A fourth example includes the method of the third example, wherein the target temperature comprises a target temperature increase within a range from approximately 0.1 degrees C. to approximately 6 degrees C.

A fifth example includes the method of any of the first through fourth examples, wherein the one or more temperature sensors comprise a plurality of temperature sensors, each of the plurality of temperature sensors configured to sense temperature of a respective portion of the other tissue, and presenting information indicating flow of heat comprises presenting information indicating flow of heat from the target point of tissue to the respective portions of the other tissue over the period of time based on the temperatures sensed by the plurality of temperature sensors over the period of time to facilitate characterization of the at least one of anatomy or function of the tissue.

A sixth example includes the method of any of the first through fifth examples, wherein the tissue comprises a three-dimensional volume of tissue comprising the target point of the tissue and the other tissue proximate to the target point.

A seventh example includes the method of any of the first through sixth examples, wherein the other tissue surrounds the target point.

An eighth example includes the method of any of the first through seventh examples, wherein delivering the ultrasound comprises delivering an ultrasound beam focused on the target point of tissue.

A ninth example includes the method of any of the first through eighth examples, further comprising: controlling, by the one or more processors, the one or more temperature sensors to sense a temperature of at least one of the target point or the other tissue during delivery of the ultrasound to the target point of tissue; and determining, by the one or more processors, whether to control the one or more ultrasound transducers to continue to deliver the ultrasound to the target point of tissue based on the temperature sensed during the delivery of the ultrasound to the target point of tissue.

A tenth example includes the method of any of the first through ninth examples, wherein presenting information indicating flow of heat comprises presenting a map indicating the flow of heat from the target point of tissue to the other tissue over the period of time based on the sensed temperatures to facilitate characterization of at least one of anatomy or function of the tissue.

An eleventh example includes the method of the tenth example, wherein presenting the map comprises presenting the map and a depiction of anatomy of the tissue in an overlayed relationship.

A twelfth example includes the method of the tenth or eleventh example, wherein the map comprises a plurality of voxels and indicates thermal diffusion at each of the plurality of voxels.

A thirteenth example includes the method of any of the first through twelfth examples, wherein the information indicating flow of heat from the target point of tissue to the other tissue over the period of time indicates an anisotropic characteristic of the tissue.

A fourteenth example includes a system comprising means for performing any of the methods of the first through thirteenth examples, the system comprising: means for delivering ultrasound to a target point of tissue of a patient to heat the target point of the tissue; means for sensing a temperature of other tissue of the patient proximate to the target point of tissue a plurality of times over a period of time after the target point of the tissue has been heated; and means for presenting information indicating flow of heat from the target point of tissue to the other tissue over the period of time based on the sensed temperatures to facilitate the characterization of at least one of anatomy or function of the tissue.

A fifteenth example includes a computer-readable storage medium comprising instructions that, when executed by one or more processors, cause the one or more processors to perform the method of any of the first through thirteenth examples, wherein the instructions cause the one or more processors to: control delivery of ultrasound by one or more ultrasound transducers to a target point of the tissue to heat the target point of the tissue; control one or more temperature sensors to sense a temperature of other tissue of the patient proximate to the target point of tissue a plurality of times over a period of time after the target point of the tissue has been heated; and present information indicating flow of heat from the target point of tissue to the other tissue over the period of time based on the sensed temperatures to facilitate the characterization of at least one of anatomy or function of the tissue.

A sixteenth example includes a device configured to deliver ultrasound to tissue of a patient, the device comprising: a flexible interconnect element; a plurality of ultrasound transducers distributed on and connected to the flexible interconnect element; one or more power sources connected to the flexible interconnect element; signal generation circuitry powered by the one or more power sources and connected to the flexible interconnect element; one or more processors powered by the one or more power sources and connected to the flexible interconnect element, wherein the one or more processors are configured to control the signal generation circuitry to apply at least one signal to a selected one or more of the plurality of ultrasound transducers and thereby control the one or more ultrasound transducers to deliver ultrasound to the tissue of the patient; and an attachment element configured to attach the device to the patient, wherein attachment element is connected to at least one of the flexible interconnect element, the plurality of ultrasound transducers, the one or more power sources, the signal generation circuitry, or the one or more processors.

A seventeenth example includes the device of the sixteenth example, wherein the flexible interconnect element comprises a flexible circuit that electrically connects at least two of: the plurality of ultrasound transducers, the one or more power sources, the signal generation circuitry, and the one or more processors

An eighteenth example includes the device of the sixteenth example or seventeenth example, wherein the plurality of ultrasound transducers are distributed on the flexible interconnect element in a two-dimensional array.

A nineteenth example includes the device of any of the sixteenth through eighteenth examples, wherein the plurality of ultrasound transducers comprises at least three ultrasound transducers.

A twentieth example includes the device of any of the sixteenth through nineteenth examples, wherein the plurality of ultrasound transducers comprises at least nine ultrasound transducers.

A twenty-first example includes the device of any of the sixteenth through twentieth examples, wherein the plurality of ultrasound transducers comprises at least thirty-two ultrasound transducers.

A twenty-second example includes the device of any of the sixteenth through twenty-first examples, wherein the plurality of ultrasound transducers comprises at least sixty-four ultrasound transducers.

A twenty-third example includes the device of any of the sixteenth through twenty-second examples, wherein the one or more power sources comprise a plurality of power sources.

A twenty-fourth example includes the device of the twenty-third example, wherein the plurality of power sources are distributed across the flexible interconnect element.

A twenty-fifth example includes the device of the twenty-third example or the twenty-fourth example, wherein the plurality of power sources are distributed across the flexible interconnect element in a two-dimensional array.

A twenty-sixth example includes the device of any of the twenty-third through twenty-fifth examples, wherein each of the plurality of power sources is associated with a respective one of the plurality of ultrasound transducers.

A twenty-seventh example includes the device of the twenty-sixth example, wherein each of the plurality of power sources is attached to the respective one of the plurality of ultrasound transducers, and configured as a backing material to tune a frequency of the respective one of the plurality of ultrasound transducers.

A twenty-eighth example includes the device of any of the twenty-third through twenty-seventh examples, wherein the plurality of power sources comprises a plurality of batteries.

A twenty-ninth example includes the device of the twenty-eighth example, wherein each of the batteries comprises a housing defining a cavity, wherein the cavity is substantially free of gas.

A thirtieth example includes the device of any of the sixteenth through twenty-ninth examples, wherein the flexible interconnect element is configured as a backing material to tune a frequency of the plurality of ultrasound transducers.

A thirty-first example includes the device of any of the sixteenth through thirtieth examples, further comprising sensing circuitry connected to one or more of the plurality of ultrasound transducers and the flexible interconnect element, wherein, for each of the one or more of the plurality of ultrasound transducers, the sensing circuitry is configured to generate a signal as a function of reflected ultrasound sensed by the ultrasound transducer.

A thirty-second example includes the device of any of the sixteenth through thirty-first examples, wherein the attachment element comprises an adhesive layer.

A thirty-third example includes the device of the thirty-second example, wherein the adhesive layer is configured as an acoustic interface between the plurality of ultrasound transducers and the tissue.

A thirty-fourth example includes the device of any of the sixteenth through thirty-third examples, further comprising a communication module connected to the flexible interconnect element, wherein the one or more processors are configured to communicate with another device via the communication module.

A thirty-fifth example includes the device of the thirty-fourth example, wherein the communication module is configured for wireless communication with the other device.

A thirty-sixth example includes the device of any of the sixteenth though thirty-fifth examples, further comprising a memory connected to the flexible interconnect element.

A thirty-seventh example includes any of the systems described herein comprising the device of any of the sixteenth through thirty-sixth examples.

Various examples have been described. These and other examples may be within the scope of the following claims.

1. A system comprising:
one or more ultrasound transducers;
one or more temperature sensors;
a user interface; and
one or more processors configured to:
control the one or more ultrasound transducers to deliver ultrasound to a target point of tissue of a patient to heat the target point of tissue;
control the one or more temperature sensors to sense a temperature of other tissue of the patient proximate to the target point of tissue a plurality of times over a period of time after the target point of tissue has been heated; and
present, via the user interface, information indicating flow of heat from the target point of tissue to the other tissue over the period of time based on the sensed temperatures to facilitate characterization of at least one of anatomy or function of the tissue.

2. The system of claim 1,
wherein the target point of tissue comprises a first target point of tissue,
wherein the one or more processors are configured to, iteratively, for each of a plurality of target points of tissue including the first target point of tissue:
control the one or more ultrasound transducers to deliver ultrasound to one of the plurality of target points of tissue to heat the target point of tissue; and
control the one or more temperature sensors to sense a temperature of other tissue of the patient proximate to the target point of tissue a plurality of times over a period of time after the target point of tissue has been heated, and
wherein the one or more processors are configured to present, via the user interface, information indicating flow of heat from the plurality of target points of tissue to the other tissue over the periods of time based on the sensed temperatures to facilitate characterization of the at least one of anatomy or function of the tissue.

3. The system of claim 1, wherein the one or more processors are configured to:
control the one or more ultrasound transducers to deliver ultrasound to the target point of tissue until the target point of tissue is heated to a target temperature; and
control the one or more temperature sensors to sense the temperature of the other tissue the plurality of times over the period of time after the target point of tissue has been heated to the target temperature.

4. The system of claim 3, wherein the target temperature comprises a target temperature increase within a range from approximately 0.1 degrees C. to approximately 6 degrees C.

5. The system of claim 1,
wherein the one or more temperature sensors comprise a plurality of temperature sensors, each of the plurality of temperature sensors configured to sense temperature of a respective portion of the other tissue, and
wherein the one or more processors are configured to present, via the user interface, information indicating flow of heat from the target point of tissue to the respective portions of the other tissue over the period of time based on the temperatures sensed by the plurality of temperature sensors over the period of time to facilitate characterization of the at least one of anatomy or function of the tissue.

6. The system of claim 1, wherein the tissue comprises a three-dimensional volume of tissue comprising the target point of tissue and the other tissue proximate to the target point.

7. The system of claim 1, wherein the other tissue surrounds the target point.

8. The system of claim 1, wherein the one or more processors are configured to control the one or more ultrasound transducers to deliver an ultrasound beam focused on the target point of tissue.

9. The system of claim 1, wherein the one or more processors are further configured to:
control the one or more temperature sensors to sense a temperature of at least one of the target point or the other tissue during delivery of the ultrasound to the target point of tissue; and
determine whether to control the one or more ultrasound transducers to continue to deliver the ultrasound to the target point of tissue based on the temperature sensed during the delivery of the ultrasound to the target point of tissue.

10. The system of claim 1, wherein the one or more processors are configured to present, via the user interface, a map indicating the flow of heat from the target point of tissue to the other tissue over the period of time based on the sensed temperatures to facilitate characterization of at least one of anatomy or function of the tissue.

11. The system of claim 10, wherein the one or more processors are configured to present the map and a depiction of anatomy of the tissue in an overlayed relationship.

12. The system of claim 10, wherein the map includes a plurality of voxels, and indicates thermal diffusion at each of the plurality of voxels.

13. The system of claim 1, wherein the information indicating flow of heat from the target point of tissue to the other tissue over the period of time indicates an anisotropic characteristic of the tissue.

14. The system of claim 1, wherein the one or more ultrasound transducers comprise a plurality of ultrasound transducers, the system further comprising a flexible device configured to be attached to the patient, wherein the flexible device comprises:
the plurality of ultrasound transducers;
at least one power source;
signal generation circuitry powered by the at least one power source; and
at least one of the one or more processors configured to control the signal generation circuitry to apply at least one signal to a selected one or more ultrasound transducers of the plurality of ultrasound transducers and thereby control the one or more ultrasound transducers to deliver ultrasound to the target point of tissue.

15. The system of claim 14, wherein the one or more temperature sensors comprise a plurality of temperature sensors, and wherein the flexible device further comprises the plurality of temperature sensors.

16. The system of claim 15, wherein the plurality of temperature sensors comprise one or more of the plurality of ultrasound transducers configured to generate a signal as a function of ultrasound reflected by the tissue, wherein the reflected ultrasound varies as a function of temperature of the tissue.

17. The system of claim 14, wherein the flexible device further comprises a communication module, the system further comprising an interface device configured to communicate with the flexible device via the communication module, wherein the interface device comprises:
at least one of the one or more processors; and
the user interface.

18. A method for facilitating characterization of at least one of anatomy or function of tissue of a patient, the method comprising:
delivering, using one or more ultrasound transducers, ultrasound to a target point of the tissue to heat the target point of the tissue;
sensing, using one or more temperature sensors, a temperature of other tissue proximate to the target point of the tissue a plurality of times over a period of time after the target point of the tissue has been heated; and
presenting, via the user interface, information indicating flow of heat from the target point of the tissue to the other tissue over the period of time based on the sensed temperatures to facilitate the characterization of at least one of anatomy or function of the tissue.

19. The method of claim 18, wherein the target point of tissue comprises a first target point of tissue, and the method comprises, iteratively, for each of a plurality of target points of tissue including the first target point of tissue:
delivering, using the one or more ultrasound transducers, ultrasound to one of the plurality of target points of tissue to heat the target point of tissue; and
sensing, using the one or more temperature sensors, a temperature of other tissue of the patient proximate to the target point of tissue a plurality of times over a period of time after the target point of tissue has been heated, and
wherein presenting information indicating flow of heat comprises presenting information indicating flow of heat from the plurality of target points of tissue to the other tissue over the periods of time based on the sensed temperatures to facilitate characterization of the at least one of anatomy or function of the tissue.

20. The method of claim 18,
wherein delivering ultrasound comprises delivering ultrasound to the target point of tissue until the target point of tissue is heated to a target temperature, and
wherein sensing temperature comprises sensing temperature of the other tissue the plurality of times over the period of time after the target point of tissue has been heated to the target temperature.

21. The method of claim 20, wherein the target temperature comprises a target temperature increase within a range from approximately 0.5 degrees to approximately 6 degrees C.

22. The method of claim 18, wherein the one or more temperature sensors comprise a plurality of temperature sensors, each of the plurality of temperature sensors configured to sense temperature of a respective portion of the other tissue, and presenting information indicating flow of heat comprises presenting information indicating flow of heat from the target point of tissue to the respective portions of the other tissue over the period of time based on the temperatures sensed by the plurality of temperature sensors over the period of time to facilitate characterization of the at least one of anatomy or function of the tissue.

23. The method of claim 18, wherein the tissue comprises a three-dimensional volume of tissue comprising the target point of the tissue and the other tissue proximate to the target point.

24. The method of claim 18, wherein the other tissue surrounds the target point.

25. The method of claim 18, wherein delivering the ultrasound comprises delivering an ultrasound beam focused on the target point of tissue.

26. The method of claim 18, further comprising:
controlling, by the one or more processors, the one or more temperature sensors to sense a temperature of at least one of the target point or the other tissue during delivery of the ultrasound to the target point of tissue; and
determining, by the one or more processors, whether to control the one or more ultrasound transducers to continue to deliver the ultrasound to the target point of tissue based on the temperature sensed during the delivery of the ultrasound to the target point of tissue.

27. The method of claim 18, wherein presenting information indicating flow of heat comprises presenting a map indicating the flow of heat from the target point of tissue to the other tissue over the period of time based on the sensed temperatures to facilitate characterization of at least one of anatomy or function of the tissue.

28. The method of claim 27, wherein presenting the map comprises presenting the map and a depiction of anatomy of the tissue in an overlayed relationship.

29. The method of claim 27, wherein the map comprises a plurality of voxels and indicates thermal diffusion at each of the plurality of voxels.

30. The method of claim 18, wherein the information indicating flow of heat from the target point of tissue to the other tissue over the period of time indicates an anisotropic characteristic of the tissue.

31. A system comprising:
means for delivering ultrasound to a target point of tissue of a patient to heat the target point of the tissue;
means for sensing a temperature of other tissue of the patient proximate to the target point of tissue a plurality of times over a period of time after the target point of the tissue has been heated; and
means for presenting information indicating flow of heat from the target point of tissue to the other tissue over the period of time based on the sensed temperatures to facilitate the characterization of at least one of anatomy or function of the tissue.

32. A computer-readable storage medium comprising instructions that, when executed by one or more processors, cause the one or more processors:
control delivery of ultrasound by one or more ultrasound transducers to a target point of the tissue to heat the target point of the tissue;
control one or more temperature sensors to sense a temperature of other tissue of the patient proximate to the target point of tissue a plurality of times over a period of time after the target point of the tissue has been heated; and
present information indicating flow of heat from the target point of tissue to the other tissue over the period of time based on the sensed temperatures to facilitate the characterization of at least one of anatomy or function of the tissue.

33. A device configured to deliver ultrasound to tissue of a patient, the device comprising:
a flexible interconnect element;
a plurality of ultrasound transducers distributed on and connected to the flexible interconnect element;
one or more power sources connected to the flexible interconnect element;
signal generation circuitry powered by the one or more power sources and connected to the flexible interconnect element;
one or more processors powered by the one or more power sources and connected to the flexible interconnect element, wherein the one or more processors are configured to control the signal generation circuitry to apply at least one signal to a selected one or more of the plurality of ultrasound transducers and thereby control the one or more ultrasound transducers to deliver ultrasound to the tissue of the patient; and
an attachment element configured to attach the device to the patient, wherein attachment element is connected to at least one of the flexible interconnect element, the plurality of ultrasound transducers, the one or more power sources, the signal generation circuitry, or the one or more processors.

34. The device of claim 33, wherein the flexible interconnect element comprises a flexible circuit that electrically connects at least two of: the plurality of ultrasound transducers, the one or more power sources, the signal generation circuitry, and the one or more processors

35. The device of claim 33, wherein the plurality of ultrasound transducers are distributed on the flexible interconnect element in a two-dimensional array.

36. The device of claim 33, wherein the one or more power sources comprise a plurality of power sources, wherein each of the plurality of power sources is associated with a respective one of the plurality of ultrasound transducers.

37. The device of claim 36, wherein each of the plurality of power sources is attached to the respective one of the plurality of ultrasound transducers, and configured as a backing material to tune a frequency of the respective one of the plurality of ultrasound transducers.

38. The device of claim 37, wherein the plurality of power sources comprises a plurality of batteries.

39. The device of claim 38, wherein each of the batteries comprises a housing defining a cavity, wherein the cavity is substantially free of gas.

40. The device of claim 33, wherein the attachment element comprises an adhesive layer, wherein the adhesive layer is configured as an acoustic interface between the plurality of ultrasound transducers and the tissue.
